(19) United States
(12) Patent Application Publication
(10) Pub. No.: US 2009/0175817 A1
(43) Pub. Date: Jul. 9, 2009

(54) Personal Care Composition

(76) Inventors: Wing Kin Li, East Brunswick, NJ (US); Susan L. Jordan, Doylestown, PA (US); Dary L. Beatty, Lake Jackson, TX (US); Xiaodong Zhang, Livington, NJ (US); Wei Hong Yu, Edison, NJ (US)

Correspondence Address: The Dow Chemical Company, Intellectual Property Section, P.O. Box 1967, Midland, MI, 48641-1967, US

(21) Appl. No.: 11/916,904

(22) PCT Filed: May 9, 2006
(86) PCT No.: PCT/US2006/017974

§ 371 Date: Aug. 8, 2008

Related U.S. Application Data
(60) Provisional application No. 60/688,407, filed on Jun. 8, 2005.

(51) Int. Cl. A61K 8/85; C08G 63/08; A61Q 19/00; A61Q 19/10; A61Q 5/02; A61Q 5/12

(52) U.S. Cl. 424/70.11; 424/78.03; 528/354

(57) ABSTRACT

A personal care composition comprising an alkylene oxide-lactone based polymer, the polymer comprising an alkylene oxide and a lactone in copolymerized form, is useful for treating hair and skin.

### FIELD OF THE INVENTION

The present invention relates to new personal care compositions, particularly hair care or skin care compositions.

### BACKGROUND OF THE INVENTION

Hair care compositions, such as shampoos, are routinely used to clean human hair to remove excess soil and sebum secreted by the scalp. However, shampooing can leave the hair in a wet, tangled, and generally unmanageable state. After drying, the hair often becomes rough, lusterless, or frizzy due to removal of the hair's natural oil and other natural conditioning and moisturizing components. A variety of approaches have been developed to alleviate these after-shampoo problems. These approaches range from post-shampoo application of hair conditioners such as leave-on and rinse-off products, to hair conditioning shampoos which attempt both to cleanse and condition the hair from a single product. A wide variety of conditioning agents, such as cationic polymers and silicone-based conditioning agents have been used. UCARE Polymers, which are commercially available from Amerchol Corporation, a subsidiary of The Dow Chemical Company, are well known cationic hair conditioning polymers used in shampoo compositions. Many shampoo compositions typically deliver good wet hair conditioning but are typically not as effective for conditioning dry hair. These shampoo compositions often do not provide sufficient positive benefits when the hair is dried, such as a moisturized feel, smooth texture or soft feel. On the other hand, shampoos comprising a silicone-based conditioning agent often provide conditioning benefits when the hair is dried, such as smooth feel, softness and shine of dried hair. However, since a silicone-based conditioning agent typically is insoluble in the shampoo, much of it is washed off the hair during rinsing. Silicone deposition onto hair can partially be controlled by the use of cationic polymers, that means positively charged molecules. Even with the help of such cationic polymers, much of the silicone-based conditioning agent is not deposited and conditioning benefits for dried hair are not achieved to a sufficient extent. Inappropriate choice and application of cationic polymers can make silicone deposition indiscriminate and uncontrolled resulting in hair that feels weighed down and overly conditioned with a resultant loss of volume. In addition, because known silicone-based conditioning agents are typically insoluble in water-based shampoos, they can only be used in opaque shampoo formulations. So far, it has not been possible to produce a clear shampoo formulation with suitable conditioning benefits for dried hair.

The International Patent Application WO 03/047540 A1 discloses a hair shampoo composition comprising at least 0.05 weight percent of a polyalkylene glycol alkyl ether with an HLB <10 with the aim to provide a long lasting moisturized feel and a smooth feel when the hair is dried, yet not to leave the hair feeling greasy, as well as to provide softness and ease of combing when the hair is wet. The polyalkylene glycol alkyl ether can be used in combination with a silicone. The polyalkylene glycol alkyl ether is insoluble in water-based hair shampoo compositions and is useful in opaque shampoos.

U.S. Pat. No. 5,837,661 discloses hair conditioning shampoos comprising a detersive surfactant, a silicone hair conditioning agent, a suspending agent and a high molecular weight polyalkylene glycol. Preferred are polyethylene glycols having from 1,500 to 25,000 degrees of ethoxylation. The U.S. patent discloses that these polyalkylene glycols enhance the spreadability of silicone-containing shampoo compositions in hair.

U.S. Pat. No. 6,451,300 discloses the incorporation of polyalkylene glycols in shampoo compositions comprising anti-dandruff particles for influencing the amount of anti-dandruff particles that are bioavailable.

In view of the ever-increasing consumer expectations, there is the constant need to provide improved personal care compositions. Accordingly, one object of the present invention is to provide a new personal care composition, particularly a new hair care or skin care composition, preferably a new hair cleansing or skin cleansing composition or a new leave-on hair care or skin care composition. A preferred object of the present invention is to provide new hair care compositions with sufficient conditioning benefits for wet hair or dry hair or both, which can be formulated as clear or as opaque formulations and which can be formulated in the presence or absence of a silicone-based component. Another preferred object of the present invention is to provide new hair care compositions with enhanced deposition of benefit agents, such as silicone-based components, on hair. Yet another preferred object of the present invention is to provide new hair care compositions which provide an improved positive feel to wet or dried hair or both. Yet another preferred object of the present invention is to provide new hair care compositions which provide improved comb-ability to wet or dried hair or both. Yet another preferred object of the present invention is to provide a new skin care composition that provides an improved moisturized and/or soft feel of skin. Yet another preferred object of the present invention is to provide a new leave-on skin care composition that provides an improved moisturizing and skin barrier effect.

### SUMMARY OF THE INVENTION

One aspect of the present invention is a personal care composition which comprises an alkylene oxide-lactone based polymer, the polymer comprising an alkylene oxide and a lactone in copolymerized form.

Another aspect of the present invention is a method of treating hair or skin by administering the personal care composition of the present invention to the hair or skin.

### DETAILED DESCRIPTION OF THE INVENTION

The alkylene oxide-lactone based polymer comprises an alkylene oxide and a lactone in copolymerized form. By the term “comprises an alkylene oxide and a lactone in copolymerized form” is meant that the polymer comprises one or more types of alkylene oxides and one or more types of lactones in copolymerized form. Most preferably, the polymer comprises one or two types of alkylene oxides and one type of lactone in copolymerized form. Alkylene oxide-lactone based polymers which comprise a lactone and an alkylene oxide in copolymerized form are known in the art, see for example U.S. Pat. Nos. 3,312,753; 3,689,531; 4,291,155; and 5,525,702, all of which are incorporated herein in their entirety by reference. None of these patents suggests incorporating the alkylene oxide-lactone based polymers in a personal care composition. U.S. Pat. No. 5,525,702 discloses the use of these polymers as nonionic surfactants and foam control agents.

The personal care composition of the present invention generally comprises from 0.01 to 10, preferably from 0.05 to 5, more preferably from 0.5 to 2 percent of the alkylene oxide-lactone based polymer, based on the total weight of the composition.

The units derived from the alkylene oxide and from the lactone can be arranged in blocks, as disclosed in U.S. Pat. Nos. 3,312,753; 3,689,531; 4,291,155 or can be arranged randomly. Random copolymers and block copolymers of one or more lactones and one or more alkylene oxides are described in U.S. Pat. No. 5,525,702. The random alkylene oxide-lactone based polymers can be uncapped or can be capped with an alkylene oxide block which comprises the same and/or one or more different alkylene oxides as that used to make the random copolymer.

The alkylene oxide-lactone based polymer preferably comprises in copolymerized form A. an alkylene oxide of the formula (I) below and B. a lactone of the formula (II) below.

The alkylene oxide A. is represented by the formula

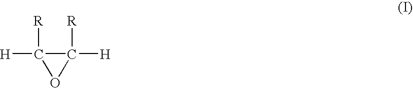

in which each R, individually, is hydrogen, a C₁-C₁₂, preferably C₁-C₆ alkyl, C₁-C₁₂, preferably C₁-C₆ haloalkyl or C₁-C₁₂, preferably C₁-C₆ alkoxy group, or in which the two R substituents together with both vicinal epoxy carbons form a saturated or monoethylenically unsaturated cycloaliphatic hydrocarbon ring, preferably of five or six carbon atoms. The preferred alkylene oxide monomers contain 2 to 12 carbon atoms, and representative alkylene oxide monomers for example include ethylene oxide, propylene oxide, the butylene oxides, 1,2-epoxydodecane, cyclopentene oxide, cyclohexene oxide and epichlorohydrin. The butylene oxides, particularly 1,2-butylene oxide, and propylene oxide are the more preferred alkylene oxide monomers. While the alkylene oxide component may comprise two or more different alkylene oxides, e.g. a mixture of ethylene oxide and propylene oxide, typically it consists of a single alkylene oxide.

The lactone B. used in this invention may be any lactone or combination of lactones having at least four carbon atoms in the ring. Preferred lactones are those represented by the formula

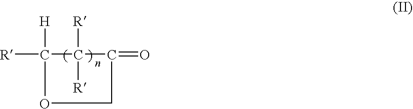

in which n is at least two, preferably from 3 to 5, and each R′ is independently hydrogen, C₁-C₈ alkyl, preferably C₁-C₄ alkyl, cyclohexyl, C₁-C₈ alkoxy, preferably C₁-C₄ alkoxy, or a single ring aromatic hydrocarbon group; with the proviso that at least four R′ are hydrogen.

The preferred lactones include unsubstituted epsilon-caprolactones, epsilon-caprolactones substituted on the carbon atoms in the ring by one, two or three C₁-C₄ alkyl groups, and unsubstituted delta-valerolactones and gamma-butyrolactones. Preferred ε-caprolactones are those of the formula

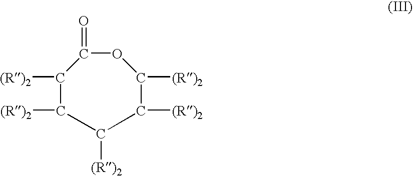

wherein each R″, independently, is hydrogen or a C₁-C₄ alkyl or C₁-C₄ alkoxy group, with the proviso that no more than three R″ substituents are groups other than hydrogen. The most preferred lactone is unsubstituted ε-caprolactone.

The preparation of lactones is well known and summarized in U.S. Pat. No. 5,525,702.

The alkylene oxide-lactone based polymer can be produced from the one or more of the above described alkylene oxides and one or more lactones in a known manner, for example as described in U.S. Pat. Nos. 3,312,753; 3,689,531; 4,291,155; and 5,525,702. Typically a chain initiator and a catalyst are used in the polymerization process. The chain initiator may be monofunctional or polyfunctional, such as di-, tri-, or tetra-functional, the functional site(s) typically comprising a reactive hydrogen. Suitable chain initiators are for example those comprising 2 or more active hydrogen atoms per molecule, for example a polyfunctional alcohol, amine, mercaptan, phenol or polycarboxylic acid, such as those described in column 4, lines 45-52 of U.S. Pat. No. 4,291,155; or organic monohydroxyl initiators, such as those described in column 4, lines 18-35 of U.S. Pat. No. 3,689,531; or the chain initiators disclosed in column 3, lines 62-67 and column 4, lines 1-29 of U.S. Pat. No. 5,525,702. Useful catalysts and reaction conditions are described in the above-mentioned patent publications.

The alkylene oxide-lactone based polymer preferably comprises from 1 to 99, more preferably from 50 to 98, most preferably from 50 to 80 mol percent of one or more alkylene oxides and from 1 to 99, more preferably from 2 to 50, most preferably from 20 to 50 mol percent of one or more lactones in copolymerized form. The weight average molecular weight of the polymer preferably is from 200 to 100,000, more preferably from 500 to 20,000, most preferably from 1000 to 5,000, in particular from 2,000 to 4,000. The weight average molecular weight is determined according to GPC (Gel Permeation Chromatography) analysis.

In one embodiment of this invention, the alkylene oxide-lactone based polymer is capped with an alkylene oxide, which can be the same but is preferably different than the alkylene oxide(s) used to form the polymer, to give a polymeric block cap. Preferably, the alkylene oxide-lactone based polymer comprises the alkylene oxide of formula I and the lactone of formula II in randomly copolymerized form and the polymer is capped with a block of polymerized alkylene oxide of Formula I, most preferably ethylene oxide. The polymeric block resulting from the capping step consists of a homopolymer if a single monomer is used, or a copolymer if more than one monomer is used. Illustrative capped polymers include (butylene oxide/lactone)-ethylene oxide and propylene oxide/lactone)-ethylene oxide polymers having a (butylene oxide/lactone) or (propylene oxide/lactone) copolymer of greater than 300, preferably from 750 to 20,000, more preferably from 750 to 4,000, weight average molecular weight with a final ethylene oxide weight percentage of greater than zero to 90, preferably 5 to 80, more preferably 10 to 40, based on the total weight average molecular weight of the polymer. In some embodiments, the capping block can include polymerized monomer units other than an alkylene oxide, such as an epsilon-caprolactone.

In another embodiment of this invention, the alkylene oxide-lactone based polymer is left uncapped.

The personal care composition of the present invention typically comprises an aqueous diluent, preferably water or an aqueous solution of one or more lower alkyl alcohols, preferably monohydric alcohols having 1 to 6 carbons, more preferably ethanol or isopropanol. Preferably, the aqueous diluent is substantially water. The pH of the personal care composition of the present invention is preferably from 4 to 9, more preferably from 4.5 to 7.5. Buffers and other pH adjusting agents can be included to achieve the desirable pH.

The personal care composition of the present invention may further comprise one or more optional components known for use in hair care or skin care products, provided that the optional components are physically and chemically compatible with the essential components described herein, or do not otherwise unduly impair product stability, aesthetics or performance. Individual concentrations of such optional components typically range from 0.001 to 10 percent by weight of the personal care compositions.

Non-limiting examples of optional components for use in the personal care composition include cationic polymers, particles, conditioning agents (such as hydrocarbon oils, fatty esters or silicones), anti dandruff agents, suspending agents, viscosity modifiers such as thickeners, dyes, nonvolatile solvents or diluents (water soluble and insoluble), pearlescent aids, foam boosters, surfactants other than alkylene oxide-lactone based polymers, pH adjusting agents, perfumes, preservatives, chelants, proteins, skin active agents, sunscreens, UV absorbers, and vitamins. Some the preferred optional components of the personal care composition of the present invention are described in more detail below.

The personal care composition of the present invention preferably comprises one or more cationic polymers in addition to the alkylene oxide-lactone based polymer. The total amount of the cationic polymer, if present, preferably is from 0.01 to 10, more preferably from 0.05 to 2, most preferably from 0.1 to 0.5 percent, based on the total weight of the composition. The weight ratio of cationic polymer to alkylene oxide-lactone based polymer preferably is from 0.01 to 1:1, more preferably from 0.1 to 0.5:1.

Suitable cationic polymers are described in the International Patent Application WO 03/047540 A1, pages 10-15, the teaching of which is incorporated herein by reference. Preferred cationic polymers have cationic charge densities of at least 0.4 meq/gm, preferably at least 0.5 meq/gm, but also preferably less than 7 meq/gm, more preferably less than 5 meq/gm, at the pH of intended use of the personal care composition. The most preferred cationic charge densities are from 0.5 to 2.1 meq/gm. The average molecular weight of such suitable cationic polymers generally is from 10,000 to 10 millions, preferably from 50,000 to 5 millions, more preferably from 100,000 to 3 millions. The “cationic charge density” of a polymer, as that term is used herein, refers to the ratio of the number of positive charges on a monomeric unit of which the polymer is comprised to the molecular weight of said monomeric unit. The cationic charge density multiplied by the polymer molecular weight determines the number of positively charged sites on a given polymer chain. The cationic polymer preferably contains cationic nitrogen-containing moieties such as quaternary ammonium or cationic protonated amino moieties. The cationic protonated amines can be primary, secondary, or tertiary amines. Known anionic counterions can be use in association with the cationic polymers, preferably halides, such as chloride, fluoride, bromide, or iodide; or sulfate or methylsulfate.

Preferred cationic polymers are quaternary nitrogen-containing polysaccharides, preferably quaternary nitrogen-containing cellulose ethers, such as those described in U.S. Pat. Nos. 3,472,840; 3,962,418; 4,663,159, and U.S. Pat. No. 5,407,919, the teaching of which is incorporated herein by reference. Particularly preferred are quaternary nitrogen-containing hydroxyethyl celluloses. Examples of preferred cationic polymers are salts of hydroxyethyl cellulose reacted with a trimethyl ammonium substituted epoxide, referred to in the industry by the Cosmetic, Toiletry, and Fragrance Association (CTFA) as Polyquaternium-10 and which are commercially available from Amerchol Corp., a subsidiary of The Dow Chemical Company, as UCARE™ Polymer JR-125, UCARE Polymer JR-400, UCARE Polymer KF, UCARE Polymer JR-30M, UCARE Polymer LR-400, UCARE Polymer LR-30M, and UCARE Polymer LK. Examples of other preferred cationic polymers are referred to by CTFA as Polyquaternium-67. They are commercially available from Amerchol Corp. as SoftCAT™ SL 5, SoftCAT SL 30, SoftCAT SL 60, SoftCAT SL 100, SoftCAT SK-L, SoftCAT SK-M, SoftCAT SK-M, SoftCAT SK-MH and SoftCAT SK-H. Other examples of preferred cationic polymers are those referred to in the industry by the CTFA as Polyquaternium-7 with the CAS Registry Number 026590-05-6, and those referred by the CTFA as Polyquaternium-44. Other suitable types of cationic cellulose ethers include the polymeric quaternary ammonium salts of hydroxyethyl cellulose reacted with lauryl dimethyl ammonium-substituted epoxide referred to in the industry (CTFA) as Polyquaternium 24. Other suitable cationic polymers include cationic guar gum derivatives and cationic starch derivatives.

The personal care composition of the present invention may comprise one or more conditioning agents. Conditioning agents include any material which is used to give a particular conditioning benefit to hair and/or skin. In hair treatment compositions, suitable conditioning agents are those which deliver one or more benefits relating to shine, softness, comb-ability, antistatic properties, wet feel, damage, manageability, body, and greasiness. In skin treatment compositions, suitable conditioning agents are those which deliver one or more benefits relating to moisturized and soft feel. The conditioning agents useful in the compositions of the present invention typically comprise a water insoluble, water dispersible, non-volatile, liquid that forms emulsified, liquid particles or are solubilized by surfactant micelles.

Preferred conditioning agents are silicone-based compounds, generally designated as silicones, such as silicone oils, cationic silicones, silicone gums, high refractive silicones, and silicone resins; or organic conditioning oils, such as hydrocarbon oils, polyolefins, and fatty esters; or combinations thereof. The total amount of these conditioning agents, if present, preferably is from 0.01 to 10, more preferably from 0.05 to 5, most preferably from 0.1 to 1.0 percent, based on the total weight of the composition. The weight ratio of such conditioning agents to the alkylene oxide-lactone based polymer in the personal care composition preferably is from 0.01 to 5:1, more preferably from 0.1 to 1:1. Suitable silicones are described in the International Patent Application WO 03/047540 A1, pages 16-26, the teaching of which is incorporated herein by reference. Suitable organic conditioning oils, such as hydrocarbon oils, polyolefins, and fatty esters are described on pages 26-29 of WO 03/047540 A1, the teaching of which is incorporated herein by reference. Preferred silicone oils include polyalkyl or polyaryl siloxanes. The aliphatic or aryl groups substituted on the siloxane chain may have any structure so long as the resulting silicones remain fluid at room temperature, are hydrophobic, are neither irritating, toxic nor otherwise harmful when applied to hair or skin. The silicone atom of each monomeric silicone unit preferably is substituted with two aliphatic or aryl groups which may represent different groups, but preferably they represent the same groups. Preferred alkyl and alkenyl substituents are C₁ to C₅, more preferably C₁ to C₄, most preferably C₁ to C₂ alkyls and alkenyls. Specific non-limiting examples of preferred silicones include: polydimethyl siloxane, polydiethylsiloxane, and polymethylphenylsiloxane. Polydimethylsiloxane is especially preferred.

Furthermore, the personal care composition of the present invention may comprise one or more surfactants. The total amount of the surfactants, if present, preferably is from 5 to 30, more preferably from 10 to 20 percent, based on the total weight of the composition. One type of suitable surfactants are the detersive surfactants described in the International Patent Application WO 03/047540 A1, pages 3-8, the teaching of which is incorporated herein by reference. Anionic, nonionic, zwitterionic, amphoteric surfactants, and mixtures thereof are preferred. Preferred surfactants include mixtures of non-ionic surfactants and anionic surfactants. Useful zwitterionic surfactants are described in U.S. Pat. No. 3,929,678.

Anionic surfactants useful herein are disclosed in U.S. Pat. No. 4,285,841, Barrat et al, issued Aug. 25, 1981, and in U.S. Pat. No. 3,919,678, Laughlin et al, issued Dec. 30, 1975. Preferred anionic surfactants include C₁₁-C₁₈ alkyl benzene sulfonates and primary or branched-chain C₁₀-C₂₀ alkyl sulfates, unsaturated sulfates such as oleyl sulfate, the C₁₀-C₁₈ alkyl alkoxy sulfates, particularly those comprising 1-7 ethoxy groups, C₁₀-C₁₈ alkyl alkoxy carboxylates, particularly those comprising 1-5 ethoxy groups, the C₁₀-C₁₈ glycerol ethers, the C₁₀-C₁₈ alkyl polyglycosides and their corresponding sulfated polyglycosides, and C₁₂-C₁₈ alpha-sulfonated fatty acid esters. Other useful anionic surfactants include water-soluble salts, particularly the alkali metal, ammonium and alkylolammonium salts, such as monoethanolammonium or triethanolammonium salts, of organic sulfuric reaction products having in their molecular structure an alkyl group containing from about 10 to about 20 carbon atoms and a sulfonic acid or sulfuric acid ester group. Other anionic surfactants useful herein are the water-soluble salts of alkyl phenol ethylene oxide ether sulfates and water-soluble salts of esters of alpha-sulfonated fatty acids. The anionic surfactants based on fatty acids include saturated and/or unsaturated fatty acids obtained from natural sources or synthetically prepared. Examples of suitable fatty acids include, but are not limited to, capric, lauric, myristic, palmitic, stearic, arachidic, and behenic acid. Other fatty acids include palmitoleic, oleic, linoleic, linolenic, and ricinoleic acid. Examples of particularly preferred surfactants are fatty acid salts, sulfonates or quaternary ammonium salts, and especially sodium lauryl sulfate (SLS) or sodium laureth sulfate (SLES).

Suitable nonionic surfactants are disclosed in U.S. Pat. No. 3,929,678, Laughlin et al., issued Dec. 30, 1975, and U.S. Pat. No. 4,285,841, Barrat et al, issued Aug. 25, 1981. Exemplary, non-limiting classes of useful nonionic surfactants include C₈-C₁₈ alkyl ethoxylates, with 1-22 ethylene oxide units and C₆-C₁₂ alkyl phenol alkoxylates, particularly ethoxylates and mixed ethoxylates/propoxylates, alkyl dialkyl amine oxides, alkanoyl glucose amides, and mixtures thereof. Other useful nonionic surfactants are polyethylene, polypropylene, and polybutylene oxide condensates of alkyl phenols. These compounds are commonly referred to as alkyl phenol alkoxylates, preferably alkyl phenol ethoxylates. Further useful nonionic surfactants are the condensation products of aliphatic alcohols with from 1 to 25 moles of ethylene oxide. The alkyl chain of the aliphatic alcohol can either be straight or branched, primary or secondary, and generally contains from 8 to 22 carbon atoms. This category of nonionic surfactant is referred to generally as “alkyl ethoxylates.” Other useful nonionic surfactants are the condensation products of ethylene oxide with a hydrophobic base formed by the condensation of propylene oxide with propylene glycol. Further useful surfactants are the condensation products of ethylene oxide with the product resulting from the reaction of propylene oxide and ethylenediamine. Fatty acid amide surfactants, C₁₂-C₁₈ betaines and sulfobetaines (sultaines) are also knows surfactants.

The personal care composition of the present invention may comprise one or more viscosity modifiers, preferably one or more polymeric thickeners. The total amount of the viscosity modifier, if present, preferably is from 0.02 to 10, more preferably from 0.5 to 2, most preferably from 0.1 to 1.0 percent, based on the total weight of the composition. Commercially available viscosity modifiers highly useful herein include Carbomers with tradenames Carbopol 934, Carbopol 940, Carbopol 950, Carbopol 980, and Carbopol 981, all available from Noveon, Inc., methylcellulose with trade name BENECEL, hydroxyethyl cellulose with trade name NATROSOL, hydroxypropyl cellulose with trade name KLUCEL, cetyl hydroxyethyl cellulose with trade name POLYSURF 67, all supplied by Hercules, ethylene oxide and/or propylene oxide based polymers with trade names CARBOWAX PEGs, POLYOX WSRs, and UCON FLUIDS, and hydroxyethyl celluloses with the trademark CELLOSIZE, all supplied by Amerchol, and methylcelluloses and hydroxypropyl methylcelluloses with the trademark METHOCEL, all supplied by The Dow Chemical Company.

The personal care composition of the present invention optionally includes particles which preferably have a particle size of less than 300 micrometers, more preferably from 0.01 to 80 micrometers. Typical particle levels are selected for the particular purpose of the composition, such as pigment particles. It has surprisingly been found that alkylene oxide-lactone based polymers provide unexpected advantages to personal care compositions, such as hair care or skin care compositions, in particular to hair cleansing or skin cleansing compositions or leave-on hair care or skin care compositions. It has unexpectedly been found that the skin care compositions of the present invention which comprise an alkylene oxide-lactone based polymer exhibit an improved moisturized and/or soft feel of skin. It has also unexpectedly been found that leave-on skin care compositions of the present invention which comprise an alkylene oxide-lactone based polymer exhibit a measurably improved moisturizing or skin barrier effect over several hours. The leave-on skin care compositions of the present invention provide long lasting moisturizing or long lasting hydration. The leave-on skin care compositions of the present invention are particularly useful in or as skin lotions, sun care lotions, lipsticks, and make up. The leave-on hair care compositions of the present invention are particularly useful in or as leave-on hair conditioners, such as instant smoothing cream. The leave-on hair care compositions of the present invention provide moisturizing without greasiness or tackiness.

It has also unexpectedly been found that the hair care compositions of the present invention which comprise an alkylene oxide-lactone based polymer exhibit an enhanced deposition of benefit agents, such as silicone-based components, on hair. Furthermore, it has unexpectedly been found that the hair care compositions of the present invention are useful for controlling the volume of the hair. For example, frizzy hair that has been treated with rinse-off hair care compositions of the present invention have a reduced volume, as compared to frizzy hair that has been treated with hair care compositions that do not comprise an alkylene oxide-lactone based polymer. Also, some consumers prefer sleek hair with a low volume. The hair care compositions of the present invention are also useful for satisfying these needs. It has also unexpectedly been found that the hair care compositions of the present invention provide an improved positive feel to wet or dried hair or improved comb-ability to wet or dried hair or combinations thereof. Furthermore, it has been found that the personal care compositions of the present invention comprising an alkylene oxide-lactone based polymer can be formulated as clear or as opaque formulations and can be formulated in the presence or absence of a silicone-based component. The compositions of the present invention are particularly useful as clear or opaque shampoo formulations, such as two-in-one formulations and antidandruff formulations. The personal care compositions of the present invention can be applied to hair or skin in a known manner. They are preferably applied to human hair or skin, but the use of the personal care compositions is not limited thereto. They can also be applied to animals, preferably mammals, such as dogs.

The present invention is further illustrated by the following examples which should not be construed to limit the scope of the present invention. Unless otherwise indicated, all parts and percentages are by weight.

### Examples 1-8 and Comparative Examples A-E

### Evaluation Method for Wet Comb-Ability

The wet combing work (WCW) is measured by using the load cell of an Instron Tensile Tester when a comb is pulled through a wet tress of European single-bleached hair available from International Hair Importers and Products Inc. The wet comb-ability of the shampoo compositions listed in Tables I and II below is calculated as follows in terms of the percent reduction in wet combing work done (% WCWD Reduction) of hair tress treated with the shampoo compositions of Examples 1 and 2 and Comparative Examples A and B in Tables I and II, as compared to hair tress treated with a Control Composition comprising 15.5 weight percent of sodium laureth-2-sulfate, 2.5 weight percent of sodium cocoamphodiacetate, citric acid (10%) to adjust the pH to 5.7 and 0.4 weight percent of GLYDANT (trademark), the remainder being water.

% WCWD Reduction=[(WCWD_{control}−WCWD_{shampooed})]/WCWD_{control}]×100

where control means that the hair tress is treated by the Control Composition described above, and

shampooed means that the hair tress is treated by a shampoo composition comprising the ingredients listed in Tables I and II below.

Evaluation of Silicone Deposition

The total amount of silicone deposited on hair that has been treated with a shampoo composition comprising a silicone oil is measured as described below. European virgin brown hair, commercially available from International Hair Importers and Products Inc. NY (USA), is used for this test. 5 gram of hair are treated with 0.5 gram of a shampoo composition listed in Table II below. The total amount of the silicone on the hair is measured in microgram silicone per gram of hair, based on the extraction with a mixture of methyl butyl ketone and toluene at a volume ratio of 1:1 and subsequent determination by Atomic Absorption Spectroscopy.

Panel Study for Wet and Dry Feel Preference and Wet and Dry Comb-Ability

A panel of 10 persons are asked to feel and comb hair tresses, in duplicate. Each panelist is asked to compare hair treated with the composition of Example 2 to the hair treated with the composition of Comparative Example B and state which tress is smoother to comb/feel. The answer “same” is not allowed.

Panel Study for Dry Skin Feel and Rinsing Properties of Body Wash Compositions

A panel of 10 persons are asked to do skin sensory studies. 0.5 grams of a body wash composition is placed on the panelist's arm and 4.5 grams of water are added. The panelists apply the product onto the volar forearm followed by rinsing. After rinsing the arm is allowed to dry for 10 minutes. Ease of rinsing and skin moisturizing feel after drying are documented and compared for samples with and without alkylene oxide-lactone based polymer.

Transepidermal Water Loss (TEWL Test)

TEWL is the imperceptible water loss through the skin, separate and distinct from active perspiration. A high TEWL indicates a compromised barrier function of the skin. In the TEWL test procedure, 50 microliters of the test formulation is applied to an area of 1 inch×1 inch (2.5 cm×2.5 cm) of volar forearm of 6 panelists. Evaporimeter tests are done after 30 minutes, 4 hours, 6 hours and 24 hours to determine water loss using a Tewameter device. The lower a result of the TEWL test is, the better is the skin barrier efficacy.

Hydration Test (Moisturization Efficacy)

Moisturizing efficacy is measured by a corneometer measurement. In this test procedure, 50 microliters of the test formulation is applied to an area of 1 inch×1 inch (2.5 cm×2.5 cm) of volar forearm of 6 panelists. The value displayed on the corneometer gives the degree of moisture on the surface of the skin before and after the skin has been treated with the skin care composition, i.e. the unit displays the status or the change in the moisture on the surface of the skin. The higher the rating of the corneometer is, the better is the moisturizing efficacy of the skin care formulation.

Components of the Hair Care and Skin Care Compositions of Examples 1-8 and Comparative Examples A-E

Alkylene oxide-lactone based polymers, prepared according to the process described in U.S. Pat. No. 5,525,702.

Copolymer 4125: A random copolymer of ε-caprolactone and propylene oxide capped with a homopolymer block of ethylene oxide. The polymer comprises 28 weight percent ε-caprolactone units, 47 weight percent propylene oxide units and 25 weight percent ethylene oxide units in polymerized form. The weight average molecular weight is 4000.

Copolymer 2187: A random copolymer of 28 weight percent ε-caprolactone and 72 weight percent propylene oxide. The weight average molecular weight is 2000.

Copolymer 4113: A random copolymer of 40 weight percent ε-caprolactone and 60 weight percent ethylene oxide. The weight average molecular weight is 2000.

Cationic Polymers:

UCARE™ Polymer JR 400: Polyquaternium-10 which is commercially available from Amerchol Corp., a subsidiary of The Dow Chemical Company, and which has a 2 percent solution viscosity of 300-500 cPs (mPa·s); and which comprises 1.5-2.2 percent nitrogen.

UCARE™ Polymer JR 30M: a cationically modified hydroxyethyl cellulose having a viscosity of 1,250-2,250 cps (mPa·s), measured as a 1 wt.-% aqueous solution, and a cationic nitrogen content of 1.5 to 2.2 percent, based on the total weight of the cellulose ether. The polymer is commercially available from Amerchol Corp.

Silicones:

A nonionic emulsion of a high molecular weight polydimethylsiloxane, commercially available as Dow Corning® 1664 Emulsion.

Surfactants: Sodium laureth-2-sulfate and sodium cocoamphodiacetate

Viscosity Modifier

A hydroxypropyl methylcellulose having a methoxyl substitution of 28-30 percent and a hydroxypropoxyl substitution of 7-12 percent and an apparent viscosity, measured as a 2 weight percent aqueous solution of about 4,000 mPa·s. It is commercially available from The Dow Chemical Company as Methocel E-4M.

Other Additives

Dimethylol dimethyl hydantoin is used as a preservative. It is recognized by the Cosmetic, Toiletry and Fragrance Association (CTFA) for use in personal care products and is commercially available under the trademark GLYDANT from LONZA Inc.

Citric acid is used as a 10 percent solution in water for pH control.

Sunflower seed oil is used as a skin moisturizing agent.

Ethylene glycol distearate is used as a opacifying agent.

Cetearyl Alcohol/Ceteareth 20 is an additive that is typically used as an emulsifying and thickening agent in lotions.

IPM, isopropyl myristate, is an emollient ester which is commercially available from Alzo International Inc. under the Trademark Dermol.

Caprylic/capric triglyceride is an emollient ester which is commercially available from Alzo International Inc. under the Trademark Dermol M-5.

The following components are used to produce clear and opaque hair and skin care compositions. All percentages in the Tables below are by weight of the composition. Water is used to complete the composition to a total of 100 percent.

TABLE I

| | | Comparative |
|---|---|---|
| | Example 1 | Example A |
| Clear Shampoo Composition: | | |
| Sodium laureth-2-sulfate | 15.5% | 15.5% |
| Sodium cocoamphodiacetate | 2.5% | 2.5% |
| UCARE ™ Polymer JR 400 | 0.5% | 0.5% |
| Copolymer 4125 | 1.0% | — |
| Citric acid (10%) | pH 5.8 | pH 5.8 |
| GLYDANT | 0.4% | 0.4% |
| Water | Remainder to 100% | Remainder to 100% |
| Properties | | |
| WCWD Reduction (%) | 64% | 19% |

TABLE II

| | | Comparative |
|---|---|---|
| | Example 2 | Example B |
| Opaque Shampoo Composition: | | |
| 2-in-1 formulation: | | |
| Sodium laureth-2-sulfate | 15.5% | 15.5% |
| Sodium cocoamphodiacetate | 2.5% | 2.5% |
| Ethylene glycol distearate | 2.0% | 2.0% |
| DOW Corning 1664 Emulsion | 1.0% | 1.0% |
| UCARE ™ Polymer JR 400 | 0.25% | 0.25% |
| Methocel E-4M | 0.5% | 0.5% |
| Copolymer 2187 | 1.0% | — |
| Citric Acid (10%) | pH 5.7 | pH 5.7 |
| GLYDANT | 0.4% | 0.4% |
| Water | Up to 100% | Up to 100% |
| Properties | | |
| WCWD Reduction (%) | 42% | 33% |
| Silicone deposition (microgram | 725 | 75 |
| silicone/gram hair) | | |
| Wet feel/Wet Comb-ability test: | | |
| Percent panelists who prefer the | 70%/70% | 30%/30% |
| listed composition | | |
| Dry feel/Dry Comb-ability test: | | |
| Percent panelists who prefer the | 100%/100% | 0%/0% |
| listed composition | | |
| Percent of panelists who | 30% | 70% |
| characterize Brazilian, | | |
| curly hair treated with the | | |
| shampoo composition as | | |
| having more volume | | |

The results in Tables I and II illustrate the unexpected benefits of an alkylene oxide-lactone based polymer in hair care compositions.

The following components are used to produce clear and opaque body wash compositions. All percentages in the tables below are by weight of the composition. Water is used to complete the composition to a total of 100 percent, based on the total composition weight.

TABLE III

| | Comparative | | |
|---|---|---|---|
| | Example C | Example 3 | Example 4 |
| Clear body wash composition | | | |
| Sodium laureth-2-sulfate | 11 | 11 | 11 |
| cocamidopropyl betaine | 4 | 4 | 4 |
| UCARE ™ Polymer JR 30M | 0.3 | 0.3 | 0.3 |
| Copolymer 2187 | 0 | 1 | 0 |
| Copolymer 4125 | 0 | 0 | 1 |
| NaCl | 0.32 | 0.32 | 0.32 |
| Water | Remainder | Remainder | Remainder |
| | to 100% | to 100% | to 100% |

| Moisturizing Properties | | |
|---|---|---|
| Percentage of panelists who prefer the composition of | 80% | — |
| Example 3 over the one of Comparative Example C | | |
| Percentage of panelists who prefer the composition of | — | 60% |
| Example 4 over the one of Comparative Example C | | |

The results in Table III illustrate that alkylene oxide-lactone based polymers provide skin care compositions with improved moisturizing feel on skin.

TABLE IV

| | Comparative | | |
|---|---|---|---|
| | Example D | Example 5 | Example 6 |
| Opaque body wash formulation | | | |
| Sodium laureth-2-sulfate | 10 | 10 | 10 |
| cocamidopropyl betaine | 3.6 | 3.6 | 3.6 |
| UCARE ™ Polymer JR 30M | 0.27 | 0.27 | 0.27 |
| Copolymer 2187 | 0 | 0.9 | 0 |
| Copolymer 4125 | 0 | 0 | 0.9 |
| NaCl | 0.29 | 0.29 | 0.29 |
| Sunflower Seed Oil | 10 | 10 | 10 |
| Water | Remainder | Remainder | Remainder |
| | to 100% | to 100% | to 100% |

| Easy to rinse | | |
|---|---|---|
| Percentage of panelists who find the composition of | 40 | — |
| Example 5 easier to rinse than that of Comp. Example D | | |
| Percentage of panelists who don't find a difference | 50 | — |
| between Example 5 and Comparative Example D | | |
| Percentage of panelists who find the composition of | — | 60 |
| Example 6 easier to rinse than that of Comp. Example D | | |
| Percentage of panelists who don't find a difference | — | 20 |
| between Example 6 and Comparative Example D | | |

The results in Table IV illustrate that most panelists find that skin care compositions comprising an alkylene oxide-lactone based polymer are easier or at least as easy to rinse as corresponding compositions that do not comprise an alkylene oxide-lactone based polymer. The difficulty to rinse off known oil-containing body wash compositions is a significant deficiency of them.

The following components are used to produce leave-on skin care products. All percentages in Table V below are by weight of the composition. Water is used to complete the composition to a total of 100 percent, based on the total composition weight.

TABLE V

| Leave-on Skin care | | | | Comparative | Comparative |
|---|---|---|---|---|---|
| formulation | | Example 7 | Example 8 | Example E | Example F |
| Cetearyl Alcohol/Ceteareth 20 | | 2.5 | 2.5 | 2.5 | 2.5 |
| Copolymer 4125 | | 5 | — | — | — |
| Copolymer 4113 | | — | 5 | — | — |
| Isopropyl myristate | | — | — | 5 | — |
| Caprylic/capric triglyceride | | 10 | 10 | 10 | 15 |
| Glycerin | | 2 | 2 | 2 | 2 |
| GLYDANT | | 0.4 | 0.4 | 0.4 | 0.4 |
| Water | | Remainder | Remainder | Remainder | Remainder |
| | | to 100% | to 100% | to 100% | to 100% |
| Hydration test by | 30 min. | 26.2 | 22.0 | 20.1 | 24.4 |
| corneometer, | 4 hours | 17.2 | 14.9 | 13.2 | 15.9 |
| measurement after: | 6 hours | 18.8 | 14.0 | 13.3 | 15.6 |
| | 24 hours | 8.5 | 10.7 | 6.8 | 6.2 |
| TEWL Test, by | 30 min. | −1.3 | 1.6 | 0.04 | 0.14 |
| Tewameter, | 4 hours | −1.54 | 0.64 | 0.32 | −0.22 |
| measurement after: | 6 hours | −0.62 | 1.4 | 0.58 | 1.54 |
| | 24 hours | −1.52 | 0.06 | 0.46 | 1.12 |

The formulations of the present invention show a better moisturization efficacy, as measured by the hydration test, than a comparative composition which does not comprise an alkylene-oxide lactone based polymer or which comprises a correspondingly higher amount of caprylic/capric triglyceride, a well-known emollient. The improved moisturizing effect of the formulations of the present invention is long lasting. The formulations of the present invention also show a better skin barrier efficacy after 24 hours, as measured with the TEWL Test, than the comparative compositions.

1. A personal care composition comprising an alkylene oxide-lactone based polymer, wherein the alkylene oxide-lactone based polymer comprises in randomly copolymerized form
A. an alkylene oxide of the formula 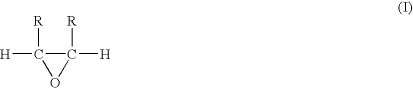
in which each R, individually, is hydrogen, a C₁-C₁₂ alkyl, C₁-C₁₂ haloalkyl or C₁-C₁₂ alkoxy group, or in which the two R substituents together with both vicinal epoxy carbons form a saturated or monoethylenically unsaturated cycloaliphatic hydrocarbon ring; and
B. a lactone of the formula 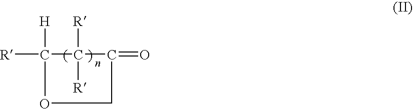
in which n is at least two and each R′ is independently hydrogen, C₁-C₈ alkyl, cyclohexyl or C₁-C₉ alkoxy; with the proviso that at least four R′ are hydrogen.

2. The personal care composition of claim 1 wherein the alkylene oxide-lactone based polymer comprises an alkylene oxide of formula I and a lactone of formula II in randomly copolymerized form and the polymer is capped with a block of polymerized alkylene oxide of Formula I.

3. The personal care composition of claim 1 wherein the alkylene oxide in the alkylene oxide-lactone based polymer is ethylene oxide, propylene oxide or a butylene oxide.

4. The personal care composition of claim 2 wherein the alkylene oxide-lactone based polymer is capped with a block of polymerized ethylene oxide.

5. The personal care composition of claim 1 wherein the lactone in the alkylene oxide-lactone based polymer is an ε-caprolactone.

6. The personal care composition of claim 5 wherein the ε-caprolactone is of the formula 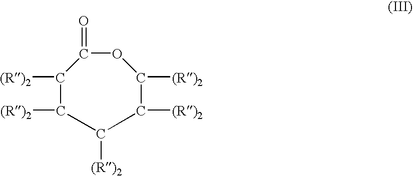
wherein each R″, independently, is hydrogen or a C₁-C₄ alkyl or C₁-C₄ alkoxy group, with the proviso that no more than three R″ substituents are groups other than hydrogen.

7. The personal care composition of claim 6 wherein the ε-caprolactone is unsubstituted ε-caprolactone.

8. The personal care composition of claim 1 comprising from 0.05 to 5 percent of the alkylene oxide-lactone based polymer, based on the total weight of the composition.

9. The personal care composition of claim 1 additionally comprising a cationic polymer.

10. The personal care composition of claim 9 comprising from 0.05 to 2 percent of the cationic polymer, based on the total weight of the composition.

11. The personal care composition of claim 1 additionally comprising a silicone.

12. The personal care composition of claim 1 in the form of a hair or skin cleansing composition.

13. The personal care composition of Claim 1 in the form of a leave-on skin care or hair care composition.

14. A method of treating hair or skin by administering the personal care composition of claim 1 to the hair or skin.

15. A method of producing a skin care composition with improved moisturized or soft feel on skin wherein an alkylene oxide-lactone based polymer is incorporated in the skin care composition and the alkylene oxide-lactone based polymer comprises in copolymerized form
A. an alkylene oxide of the formula 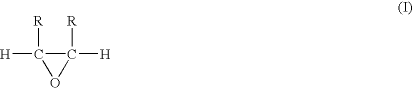
in which each R, individually, is hydrogen, a C₁-C₁₂ alkyl, C₁-C₁₂ haloalkyl or C₁-C₁₂ alkoxy group, or in which the two R substituents together with both vicinal epoxy carbons form a saturated or monoethylenically unsaturated cycloaliphatic hydrocarbon ring; and
B. a lactone of the formula 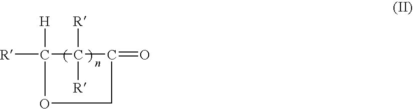
in which n is at least two and each R′ is independently hydrogen, C₁-C₈ alkyl, cyclohexyl or C₁-C₈ alkoxy; with the proviso that at least four R′ are hydrogen.

16. The method of claim 15 of producing a leave-on skin care composition with an improved moisturizing and skin barrier effect.

17. The method of claim 15 wherein the alkylene oxide-lactone based polymer set forth in claim 1 is incorporated in the skin care composition.

18. A method of producing a hair care composition that exhibits enhanced deposition of a benefit agent on hair, is useful for controlling the volume of hair or provides improved comb-ability to hair wherein an alkylene oxide-lactone based polymer is incorporated in the hair care composition and the alkylene oxide-lactone based polymer comprises in copolymerized form
A. an alkylene oxide of the formula 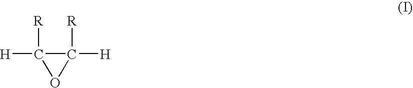
in which each R, individually, is hydrogen, a C₁-C₁₂ alkyl, C₁-C₁₂ haloalkyl or C₁-C₁₂ alkoxy group, or in which the two R substituents together with both vicinal epoxy carbons form a saturated or monoethylenically unsaturated cycloaliphatic hydrocarbon ring; and
B. a lactone of the formula 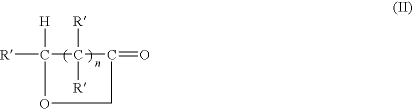
in which n is at least two and each R′ is independently hydrogen, C₁-C₈ alkyl, cyclohexyl or C₁-C₈ alkoxy; with the proviso that at least four R′ are hydrogen.

19. The method of claim 18 wherein the alkylene oxide-lactone based polymer set forth in claim 1 is incorporated in the hair care composition.
